(19) United States
(12) Patent Application Publication
(10) Pub. No.: US 2023/0092149 A1
(43) Pub. Date: Mar. 23, 2023

(54) FIBROTIC TISSUE TREATMENT DEVICE AND METHOD OF USE

(71) Applicant: MM Collective, LLC, Dallas, TX (US)

(72) Inventors: Matthew David Moulds, Lewisville, TX (US); Kelly Shawn Wood, Wichita, KS (US)

(73) Assignee: MM Collective, LLC, Dallas, TX (US)

(21) Appl. No.: 17/939,113

(22) Filed: Sep. 7, 2022

Related U.S. Application Data
(60) Provisional application No. 63/243,447, filed on Sep. 13, 2021.

(51) Int. Cl. A61B 17/14

(52) U.S. Cl. CPC A61B 17/144

(57) ABSTRACT

A device for treating fibrotic tissue, comprising a base formed from a solid material, a plurality of first teeth formed in the base, each of the plurality of first teeth separated from at least one other first tooth by a groove, a handle disposed on the base opposite the plurality of teeth, the handle having a first gripping surface and a second gripping surface disposed adjacent to the first gripping surface and a plurality of second teeth formed in the base and opposite from the second gripping surface, each of the plurality of second teeth separated from at least one other second tooth by a groove.

### RELATED APPLICATIONS

This application claims benefit of and priority to U.S. Provisional patent application No. 63/243,447, filed Sep. 13, 2021, which is hereby incorporated by reference for all purposes as if set forth herein in its entirety.

### TECHNICAL FIELD

The present disclosure relates generally to treatment of fibrotic tissue, and more specifically to a device for treating fibrotic tissue and a method of using the device.

### BACKGROUND OF THE INVENTION

Fibrotic tissue can cause pain and can also be unattractive. However, the only effective way to currently remove such fibrotic tissue is surgical, which can create additional fibrotic tissue, or by laser treatments, which on treat the superficial layers leaving fibrotic tissue to continue growing.

### SUMMARY OF THE INVENTION

A device for treating fibrotic tissue is disclosed that includes a base formed from a solid material, a plurality of first teeth formed in the base, each of the plurality of first teeth separated from at least one other first tooth by a groove, a handle disposed on the base opposite the plurality of teeth, the handle having a first gripping surface and a second gripping surface disposed adjacent to the first gripping surface and a plurality of second teeth formed in the base and opposite from the second gripping surface, each of the plurality of second teeth separated from at least one other second tooth by a groove.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a diagram of a fibrotic treatment tool with square features and a convex edge, in accordance with an example embodiment of the present disclosure;

FIG. 2 is a diagram of a fibrotic treatment tool with square features and a concave edge, in accordance with an example embodiment of the present disclosure;

FIG. 3 is a diagram of a fibrotic treatment tool with triangular features and a concave edge, in accordance with an example embodiment of the present disclosure;

FIG. 4 is a diagram of a fibrotic treatment tool with triangular features and a convex edge, in accordance with an example embodiment of the present disclosure;

FIGS. 5A-5B are data from an ultrasound examination of fibrotic tissue before and after treatment; and

FIGS. 6A-6C are data from an examination of fibrotic before and after treatment.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

Tissue that has scarred or otherwise developed fibrotic structures can cause pain and may reasonably be considered unattractive, such that treatment of such tissue is not cosmetic but medically necessary as part of the wound treatment process. Nevertheless, there are few nonsurgical options for treating such fibrotic tissue. One option is referred to as the Graston tool, which has a flat edge to apply a downward force in one direction, and which provides minimal improvement. The amount of force used with the Graston tool was not defined, and resulted in painful treatments at excessive force that did not improve the wound treatment and recovery process. The amount of force in the prior art treatments was never correlated with results. The present disclosure recognizes that in order to effectively change fibrotic tissue, it is necessary to force in more than one axis, and that doing so can greatly increase the effectiveness of treatment at relieving pain and recovering from the wound.

In order to effectively provide force to the fibrotic tissue in more than one axis, an irregular edge shape can be used that is completely unlike anything previously used. In addition to this novel and non-obvious edge shape, it is also necessary to control the amount of force provided, unlike the prior art. In general, sufficient force to pucker the tissue and perceive the opposing teeth is applied but no more, such as two to four pounds of force. In one example embodiment, before and after measurements of hardness, ultrasound, voltage, Durometer or other suitable metrics can be used to make a determination as to whether the level of force is sufficient, or if it should be increased or decreased.

When the correct level of force is applied using a treatment device that is properly configured to apply force in more than one axis, even fibrotic tissue that has been established can be treated, unlike prior art treatment devices and processes. In one example, fibrotic tissue on a patient's throat that was 40 years old which had a ropey appearance and which caused burning and tingling pain improved after 30 minutes of treatment in accordance with the present disclosure, with no returning symptoms after 4 months.

In another example, a Durometer hardness level of 16.5 was reduced for a patient to 8 after the first treatment, resulting in no returning symptoms. Typically, though, Durometer hardness levels ranging from 2.0 to 3.5 are observed prior to treatment and are reduced to 0.0 to 0.5 after one treatment.

In addition to a reduction in Durometer hardness or other such objective criteria, a change in skin structure can also be used to determine whether a treatment is effective. One patient had inward dimpling that was reduced after treatment.

Because the treatment devices of the present disclosure apply force in more than one axis, the control of the applied force is important to prevent tissue damage. As such, instead of applying more force to control treatment, the type of tool and the duration of treatment can be altered to improve effectiveness. In one example embodiment, the treatment periods and force levels in the following table can be used as part of the iterative process to determine the appropriate tool and treatment level for treating fibrotic tissue:

| Hardness Level | Duration of treatment per square inch | | Force applied | |
|---|---|---|---|---|
| 1-4 | 2.5 | Minutes | 2-3 | lbs. |
| 4-10 | 5 | Minutes | 3-4 | lbs. |
| 10-20+ | 8+ | Minutes | 4 | lbs. |

In another example embodiment, the following table can be used as part of the iterative process to determine the appropriate tool and treatment level for treating fibrotic tissue:

| Surgical site | Duration of | | | |
|---|---|---|---|---|
| Quality Level during | treatment per square | | | |
| palpation assessment | inch | | Force applied | |
| Ropey | 5 | Minutes | 2-3 | lbs. |
| Cut, clear | 5 | Minutes | 2-3 | lbs. |
| separation of | | | | |
| fascial layers not | | | | |
| meshing to opposing | | | | |
| sides | | | | |
| Lack of tissue | 2 | Minutes | 2 | lbs. |
| strength | | | | |
| Adhered to bone, | 10 | Minutes | 3-4 | lbs. |
| organ, or cavity | | | | |
| Taut | 5 | Minutes | 2-4 | lbs. |

| | | |
|---|---|---|
| Burns | 6-10 minutes (depends on | 2-4 lbs (depends on |
| | severity of burns) | severity of burns) |

With a Durometer tool, practitioners can test for an objective hardness level by pressing the device into the target of fibrotic tissue. The Durometer tool has a pin tip that is pressed against the skin, and generates a reading as a function of the level of force that is required to depress the pin tip. With ultrasound, images of tissue under the skin before and after treatment can be obtained, with measurements at ˜2.5 cm pre-treatment and zoomed in to ˜2.1 cm to 1 cm for post treatment, although other suitable ranges can also or alternatively be used as a function of the tissue being treated. The practitioner can use the same force applied pre- and post-treatment for images, or can vary the force as needed. For voltage measurements, a Tennant BioModulator available from Senergy of Irving, Tex. or other suitable equipment can be used to measure voltages per square inch, where the practitioner can contact the skin with the device and reads the unit no pressure applied or at other suitable locations.

In addition to applying pressure using the fibrotic tissue treatment device of the present disclosure, which causes force to be applied to the fibrotic tissue along two axes, the practitioner can apply a rotational clockwise and/or counterclockwise motion, to apply force in three axes, also referred to as palpation assessment. The application of three axes of force to the fibrotic tissue can also identify differences in resistance that provide additional information to the practitioner to allow them to determine the direction of rigidity, ropiness, or irregularity in the tissue, so as to customize the treatment of the tissue. The appearance of the fibrotic tissue at the surface of the skin might not reflect the condition of the fibrotic tissue underneath the surface of the skin, due to tools used in surgery, infections and other variables. Palpation assessment can be performed before, during, and after treatment and can be used to determine or modify the methods and duration of treatment. To perform palpation assessment, a practitioner moves the tissue in different directions, with or without torsional force being applied.

Durometer testing can be used to determine whether there has been a change in hardness level before and after treatment, as well as to determine whether treatment results have changed at follow-up. Ultrasound imaging can also be used to determine whether observable changes have occurred before and after treatment, as well as to determine whether treatment results have changed at follow-up, such as if there has been a development of more fibrosis. Voltage measurements can also be used to determine whether a change in voltage levels has occurred in targeted region before and after treatment as well as retained treatment results at follow-up.

| Duration of | | Pre- | Post- | Pre- | Post- | Force |
|---|---|---|---|---|---|---|
| treatment | | Voltage | Voltage | Hardness | Hardness | Applied |
| 10 | Minutes | 50, 37, | 50, 34, | 3.5 | .5 | 3 lbs. |
| | | 40, 45 | 32, 42 | | | |
| 5 | Minutes | 52, 42, | 37, 40, | 1 | .5 | 2 lbs. |
| | | 45, 48 | 37, 33 | | | |
| 7 | Minutes | 16, 21, | 18, 16, | 1.5 | 0 | 3 lbs. |
| | | 15, 19 | 16, 16 | | | |
| 10 | Minutes | 36, 34, | 41, 42, | 1.5 | 1.0 | 3 lbs. |
| | | 34, 32 | 42, 44 | | | |
| 5 | Minutes | 38, 48 | 27, 34 | 2 | .5 | 2 lbs. |

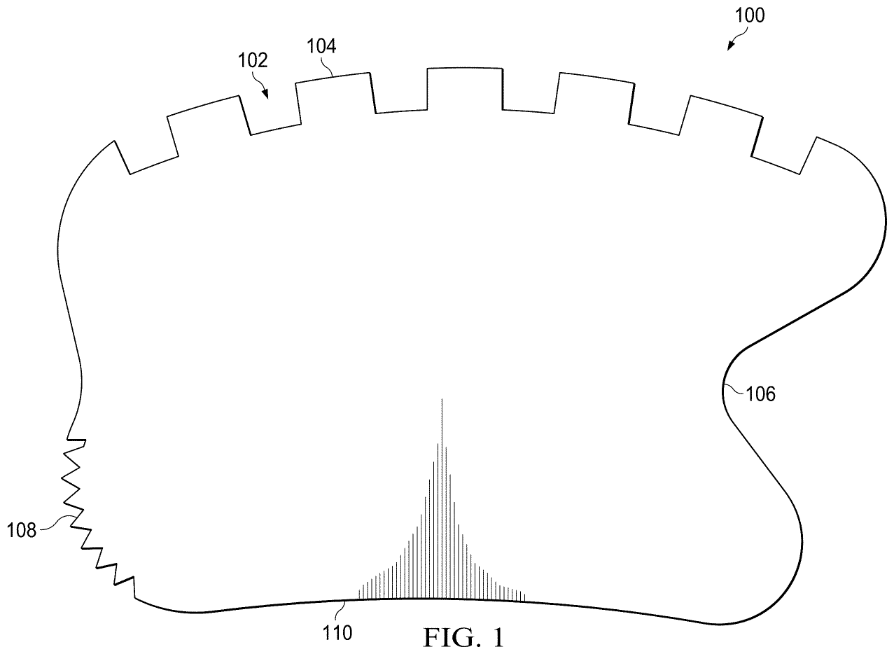

FIG. 1 is a diagram of a fibrotic treatment tool 100 with square features and a convex edge, in accordance with an example embodiment of the present disclosure. Fibrotic treatment tool 100 can be fabricated from metal, polymer, composite materials, natural materials or other suitable materials.

Fibrotic treatment tool 100 includes rectangular grooves 102, rectangular teeth 104, side grip 106, rear grip 110 and fine treatment tool 108, each of which can be fabricated by cutting, molding, machining, casting or in other suitable manners. In one example embodiment, the size of each of rectangular grooves 102, rectangular teeth 104, side grip 106, rear grip 110 and fine treatment tool 108 can be varied, such as to use alternating sizes of rectangular grooves 102, alternating sizes of rectangular teeth 104, variable sizes of rectangular grooves 102, variable sizes of rectangular teeth 104, different combinations of sizes of rectangular grooves 102 and rectangular teeth 104 and other suitable variations to improve the ability to apply force to tissue along multiple axes. Side grip 106 and rear grip 110 can likewise be located in other suitable locations, can include one or more additional materials to improve function (such as a polymer layer over a metal base), can be adjustable, can be removable or can have other suitable designs. Fine treatment tool 108 can likewise be varied in size, location, material or in other suitable manners to improve the ability to focus treatment on smaller areas without the need to replace the fibrotic treatment tool 100 in the middle of a therapy session.

Fibrotic treatment tool 100 can also include one or more sensors, such as a voltage sensor, hardness sensor, pressure sensor or other suitable sensors, to allow a user to receive treatment data in real time and while treatment is being provided. An associated sensor reader can be provided with one or more set points and associated audible or visible warning devices, to alert the user to an application of excessive or insufficient force, an expiration of a recommended treatment time or other suitable indications to assist with treatment.

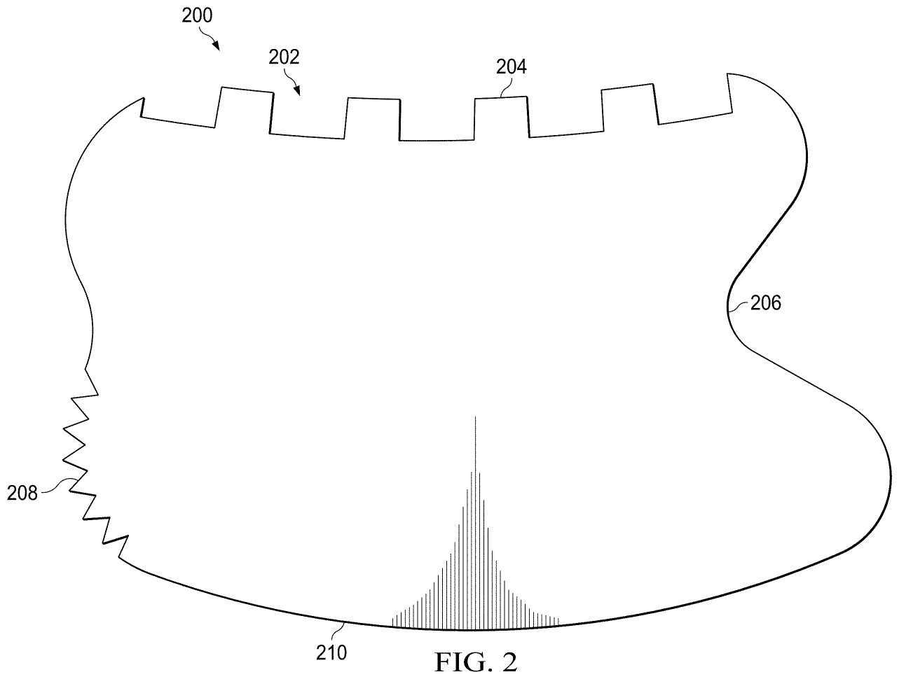

FIG. 2 is a diagram of a fibrotic treatment tool 200 with square features and a concave edge, in accordance with an example embodiment of the present disclosure. Fibrotic treatment tool 200 can be fabricated from metal, polymer, composite materials, natural materials or other suitable materials.

Fibrotic treatment tool 200 includes rectangular grooves 202, rectangular teeth 204, side grip 206, rear grip 210 and fine treatment tool 208, each of which can be fabricated by cutting, molding, machining, casting or in other suitable manners. In one example embodiment, the size of each of rectangular grooves 202, rectangular teeth 204, side grip 206, rear grip 210 and fine treatment tool 208 can be varied, such as to use alternating sizes of rectangular grooves 202, alternating sizes of rectangular teeth 204, variable sizes of rectangular grooves 202, variable sizes of rectangular teeth 204, different combinations of sizes of rectangular grooves 202 and rectangular teeth 204 and other suitable variations, such as combinations of rectangular and triangular grooves and teeth and grooves and teeth having other suitable shapes or a straight edge, to improve the ability to apply force to tissue along multiple axes. Side grip 206 and rear grip 210 can likewise be located in other suitable locations, can include one or more additional materials to improve function (such as a polymer layer over a metal base), can be adjustable, can be removable or can have other suitable designs. Fine treatment tool 208 can likewise be varied in size, location, material or in other suitable manners to improve the ability to focus treatment on smaller areas without the need to replace the fibrotic treatment tool 200 in the middle of a therapy session.

Fibrotic treatment tool 200 can also include one or more sensors, such as a voltage sensor, hardness sensor, pressure sensor or other suitable sensors, to allow a user to receive treatment data in real time and while treatment is being provided. An associated sensor reader can be provided with one or more set points and associated audible or visible warning devices, to alert the user to an application of excessive or insufficient force, an expiration of a recommended treatment time or other suitable indications to assist with treatment.

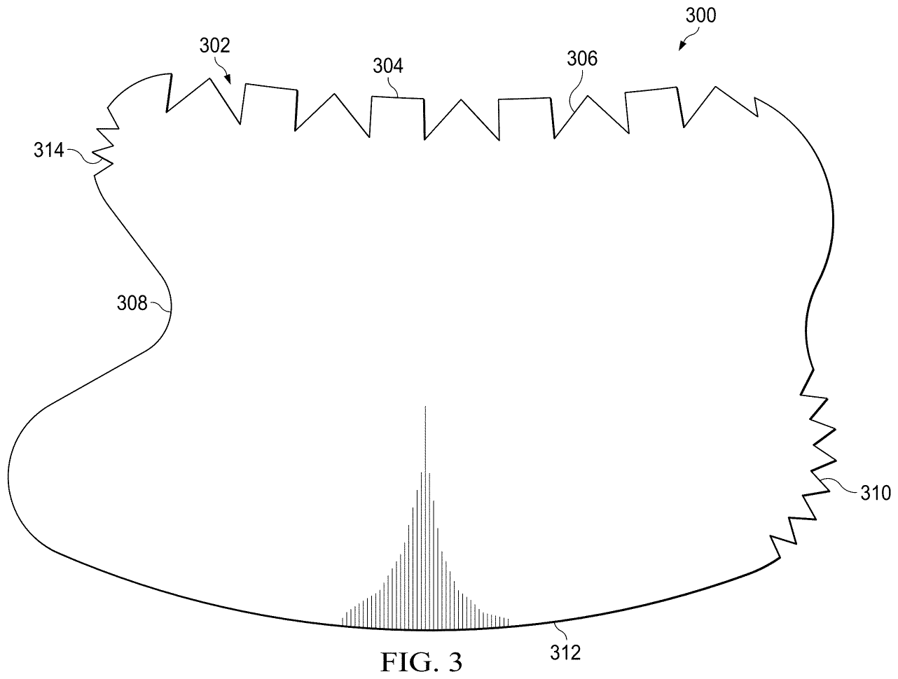

FIG. 3 is a diagram of a fibrotic treatment tool 300 with triangular features and a concave edge, in accordance with an example embodiment of the present disclosure. Fibrotic treatment tool 300 can be fabricated from metal, polymer, composite materials, natural materials or other suitable materials.

Fibrotic treatment tool 300 includes triangular grooves 302, rectangular teeth 304, triangular teeth 306, side grip 308, rear grip 312 and fine treatment tools 310 and 314, each of which can be fabricated by cutting, molding, machining, casting or in other suitable manners. In one example embodiment, the size of each of triangular grooves 302, rectangular teeth 304, triangular teeth 306, side grip 308, rear grip 312 and fine treatment tools 310 and 314 can be varied, such as to use alternating sizes of triangular grooves 302, alternating sizes of rectangular teeth 304, alternating sizes of triangular teeth 306, variable sizes of triangular grooves 302, variable sizes of triangular teeth 306, different combinations of sizes of triangular grooves 302 and triangular teeth 306 and other suitable variations, such as combinations of rectangular and triangular grooves and teeth and grooves and teeth having other suitable shapes or a straight edge, to improve the ability to apply force to tissue along multiple axes. Side grip 308 and rear grip 312 can likewise be located in other suitable locations, can include one or more additional materials to improve function (such as a polymer layer over a metal base), can be adjustable, can be removable or can have other suitable designs. Fine treatment tools 310 and 314 can likewise be varied in size, location, material or in other suitable manners to improve the ability to focus treatment on smaller areas without the need to replace the fibrotic treatment tool 300 in the middle of a therapy session.

Fibrotic treatment tool 300 can also include one or more sensors, such as a voltage sensor, hardness sensor, pressure sensor or other suitable sensors, to allow a user to receive treatment data in real time and while treatment is being provided. An associated sensor reader can be provided with one or more set points and associated audible or visible warning devices, to alert the user to an application of excessive or insufficient force, an expiration of a recommended treatment time or other suitable indications to assist with treatment.

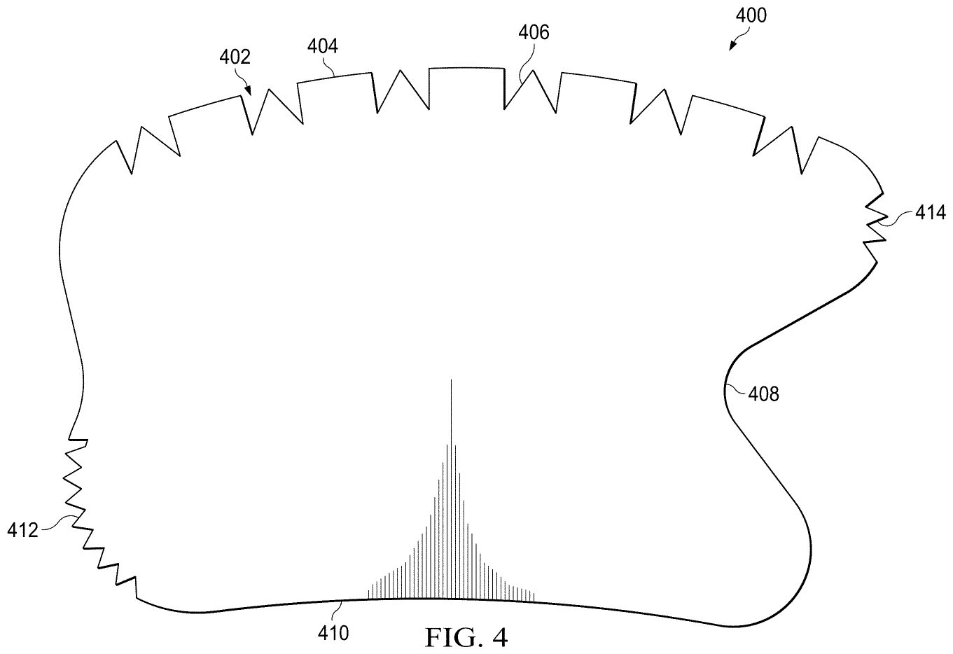

FIG. 4 is a diagram of a fibrotic treatment tool 400 with triangular features and a convex edge, in accordance with an example embodiment of the present disclosure. Fibrotic treatment tool 400 can be fabricated from metal, polymer, composite materials, natural materials or other suitable materials.

Fibrotic treatment tool 400 includes triangular grooves 402, rectangular teeth 404, triangular teeth 406, side grip 408, rear grip 412 and fine treatment tools 410 and 414, each of which can be fabricated by cutting, molding, machining, casting or in other suitable manners. In one example embodiment, the size of each of triangular grooves 402, rectangular teeth 404, triangular teeth 406, side grip 408, rear grip 412 and fine treatment tools 410 and 414 can be varied, such as to use alternating sizes of triangular grooves 402, alternating sizes of rectangular teeth 404, alternating sizes of triangular teeth 406, variable sizes of triangular grooves 402, variable sizes of triangular teeth 406, different combinations of sizes of triangular grooves 402 and triangular teeth 406 and other suitable variations, such as combinations of rectangular and triangular grooves and teeth and grooves and teeth having other suitable shapes or a straight edge, to improve the ability to apply force to tissue along multiple axes. Side grip 408 and rear grip 412 can likewise be located in other suitable locations, can include one or more additional materials to improve function (such as a polymer layer over a metal base), can be adjustable, can be removable or can have other suitable designs. Fine treatment tools 410 and 414 can likewise be varied in size, location, material or in other suitable manners to improve the ability to focus treatment on smaller areas without the need to replace the fibrotic treatment tool 400 in the middle of a therapy session.

Fibrotic treatment tool 400 can also include one or more sensors, such as a voltage sensor, hardness sensor, pressure sensor or other suitable sensors, to allow a user to receive treatment data in real time and while treatment is being provided. An associated sensor reader can be provided with one or more set points and associated audible or visible warning devices, to alert the user to an application of excessive or insufficient force, an expiration of a recommended treatment time or other suitable indications to assist with treatment.

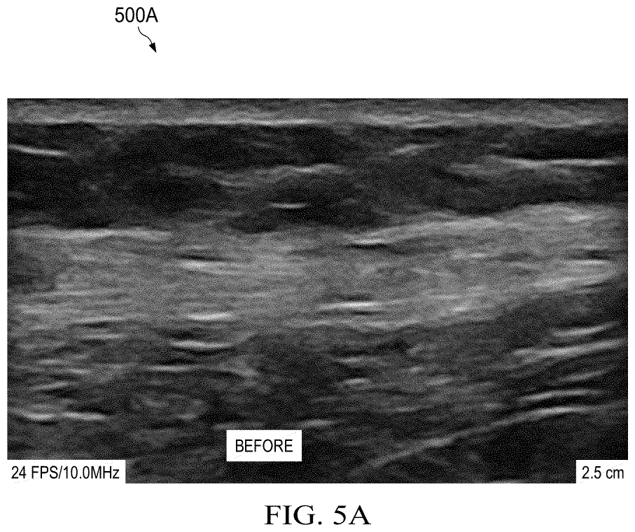

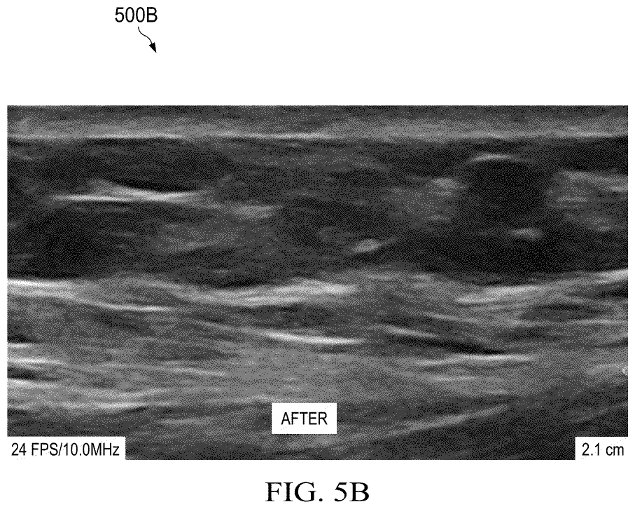

FIGS. 5A and 5B are data 500A and 500B from an ultrasound examination of fibrotic tissue before and after treatment. The before data 500A shows separation in the fibrotic tissue. The treatment was performed for 10 minutes without rotation. The after data 500B shows a small amount of change.

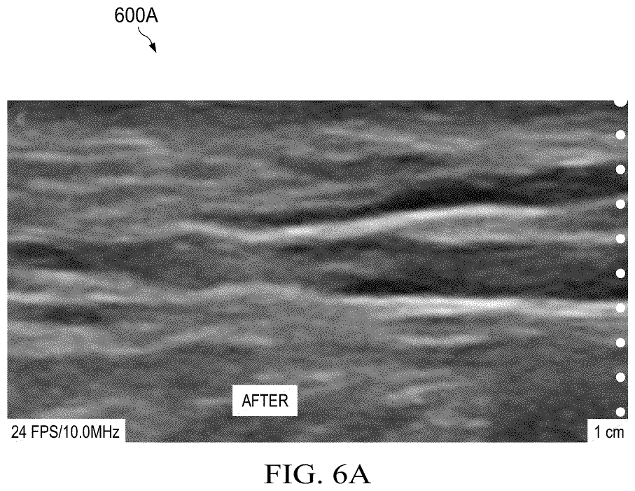

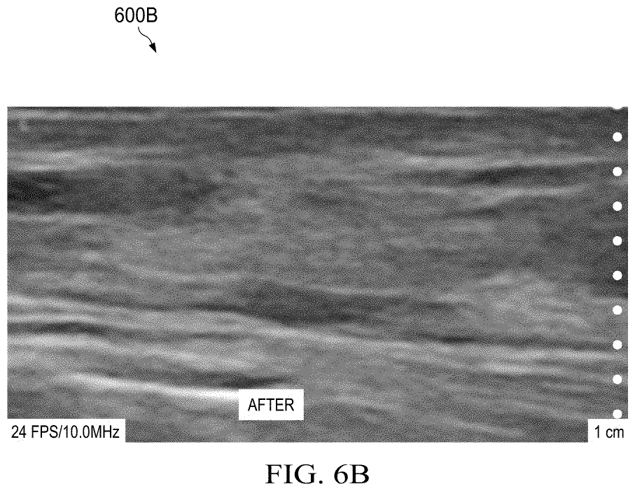

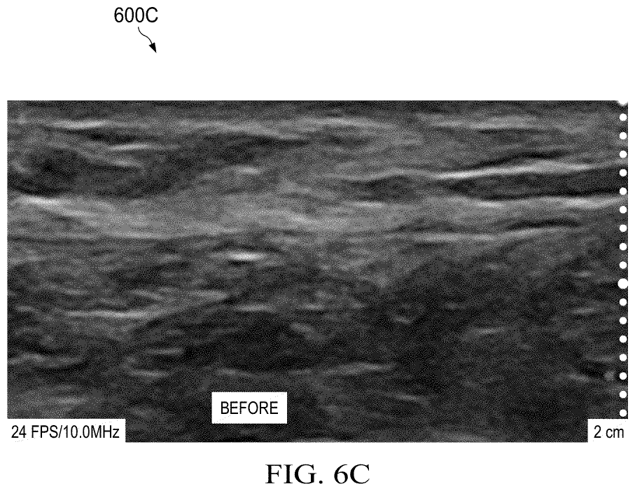

FIGS. 6A, 6B and 6C are data 600A, 600B and 600C from an examination of fibrotic before and after treatment. The before data 600A correlates to a hardness level of 3.5 and the after data 600B and 600C correlates to a hardness level of 0 after one treatment. The treatment was performed for 10 minutes with rotation.

In one example embodiment, a device for treating fibrotic tissue is disclosed that includes a base formed from a solid material, a plurality of first teeth formed in the base, each of the plurality of first teeth separated from at least one other first tooth by a first groove, a handle disposed on the base opposite the plurality of teeth, the handle having a first gripping surface and a second gripping surface disposed adjacent to the first gripping surface, a plurality of second teeth formed in the base and opposite from the second gripping surface, each of the plurality of second teeth separated from at least one other second tooth by a second groove. In another example embodiment, at least one first tooth includes at least one right angle. In another example embodiment, at least one first groove includes at least one right angle. In another example embodiment, at least one second tooth includes at least one acute angle. In another example embodiment, at least one second groove includes at least one acute angle. In another example embodiment, at least one first tooth includes at least one right angle and at least one first tooth includes at least one acute angle. In another example embodiment, at least one first groove includes at least one right angle and at least one first groove includes at least one acute angle. In another example embodiment, the device comprises a plurality of third teeth formed in the base and separate from the plurality of first teeth and the plurality of second teeth. In another example embodiment, at least one third tooth includes at least one acute angle.

In another example embodiment, a method for treating fibrotic tissue is disclosed that includes applying a force to fibrotic tissue through a base of a handheld device formed from a solid material, moving a plurality of first teeth formed in the base over the fibrotic tissue, each of the plurality of first teeth separated from at least one other first tooth by a first groove, relocating the position of the handheld device on the fibrotic tissue in relation to a handle disposed on the base opposite the plurality of teeth, the handle having a first gripping surface and a second gripping surface disposed adjacent to the first gripping surface and moving a plurality of second teeth formed in the base and opposite from the second gripping surface over the fibrotic tissue, each of the plurality of second teeth separated from at least one other second tooth by a second groove.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A device for treating fibrotic tissue, comprising:
a base formed from a solid material;
a plurality of first teeth formed in the base, each of the plurality of first teeth separated from at least one other first tooth by a first groove;
a handle disposed on the base opposite the plurality of teeth, the handle having a first gripping surface and a second gripping surface disposed adjacent to the first gripping surface; and
a plurality of second teeth formed in the base and opposite from the second gripping surface, each of the plurality of second teeth separated from at least one other second tooth by a second groove.

2. The device of claim 1, wherein at least one first tooth includes at least one right angle.

3. The device of claim 1, wherein at least one first groove includes at least one right angle.

4. The device of claim 1, wherein at least one second tooth includes at least one acute angle.

5. The device of claim 1, wherein at least one second groove includes at least one acute angle.

6. The device of claim 1, wherein at least one first tooth includes at least one right angle and at least one first tooth includes at least one acute angle.

7. The device of claim 1, wherein at least one first groove includes at least one right angle and at least one first groove includes at least one acute angle.

8. The device of claim 1, further comprising a plurality of third teeth formed in the base and separate from the plurality of first teeth and the plurality of second teeth.

9. The device of claim 1, wherein at least one third tooth includes at least one acute angle.

10. A method for treating fibrotic tissue, comprising:
applying a force to fibrotic tissue through a base of a handheld device formed from a solid material;
moving a plurality of first teeth formed in the base over the fibrotic tissue, each of the plurality of first teeth separated from at least one other first tooth by a first groove;
relocating the position of the handheld device on the fibrotic tissue in relation to a handle disposed on the base opposite the plurality of teeth, the handle having a first gripping surface and a second gripping surface disposed adjacent to the first gripping surface; and
moving a plurality of second teeth formed in the base and opposite from the second gripping surface over the fibrotic tissue, each of the plurality of second teeth separated from at least one other second tooth by a second groove.

11. The method of claim 10, wherein at least one first tooth includes at least one right angle.

12. The method of claim 10, wherein at least one first groove includes at least one right angle.

13. The method of claim 10, wherein at least one second tooth includes at least one acute angle.

14. The method of claim 10, wherein at least one second groove includes at least one acute angle.

15. The method of claim 10, wherein at least one first tooth includes at least one right angle and at least one first tooth includes at least one acute angle.

16. The method of claim 10, wherein at least one first groove includes at least one right angle and at least one first groove includes at least one acute angle.

17. The method of claim 10, further comprising moving a plurality of third teeth formed in the base and separate from the plurality of first teeth and the plurality of second teeth over the fibrotic tissue.

18. The method of claim 10, wherein at least one third tooth includes at least one acute angle.

19. In a device for treating fibrotic tissue having a base formed from a solid material, a plurality of first teeth formed in the base, each of the plurality of first teeth separated from at least one other first tooth by a first groove, a handle disposed on the base opposite the plurality of teeth, the handle having a first gripping surface and a second gripping surface disposed adjacent to the first gripping surface, a plurality of second teeth formed in the base and opposite from the second gripping surface, each of the plurality of second teeth separated from at least one other second tooth by a second groove, wherein at least one first tooth includes at least one right angle, wherein at least one first groove includes at least one right angle, wherein at least one second tooth includes at least one acute angle, wherein at least one second groove includes at least one acute angle, wherein at least one first tooth includes at least one right angle and at least one first tooth includes at least one acute angle, wherein at least one first groove includes at least one right angle and at least one first groove includes at least one acute angle, further comprising a plurality of third teeth formed in the base and separate from the plurality of first teeth and the plurality of second teeth, wherein at least one third tooth includes at least one acute angle, a method comprising:
applying a force to fibrotic tissue through a base of a handheld device formed from a solid material;
moving a plurality of first teeth formed in the base over the fibrotic tissue, each of the plurality of first teeth separated from at least one other first tooth by a first groove;
relocating the position of the handheld device on the fibrotic tissue in relation to a handle disposed on the base opposite the plurality of teeth, the handle having a first gripping surface and a second gripping surface disposed adjacent to the first gripping surface;
moving a plurality of second teeth formed in the base and opposite from the second gripping surface over the fibrotic tissue, each of the plurality of second teeth separated from at least one other second tooth by a second groove;
wherein at least one first tooth includes at least one right angle;
wherein at least one first groove includes at least one right angle;
wherein at least one second tooth includes at least one acute angle;
wherein at least one second groove includes at least one acute angle;
wherein at least one first tooth includes at least one right angle and at least one first tooth includes at least one acute angle;
wherein at least one first groove includes at least one right angle and at least one first groove includes at least one acute angle;
moving a plurality of third teeth formed in the base and separate from the plurality of first teeth and the plurality of second teeth over the fibrotic tissue; and
wherein at least one third tooth includes at least one acute angle.
